(19) United States
(12) Patent Application Publication
(10) Pub. No.: US 2019/0254852 A1
(43) Pub. Date: Aug. 22, 2019

(54) SPORTS GARMENT

(71) Applicant: PYTHON PERFORMANCE LIMITED, Edgbaston, Birmingham, West Midlands (GB)

(72) Inventor: Oscar RYNDZIEWICZ, Birmingham (GB)

(21) Appl. No.: 16/346,332

(22) PCT Filed: Nov. 1, 2017
(86) PCT No.: PCT/GB2017/053290

§ 371 Date: Apr. 30, 2019

(30) Foreign Application Priority Data
Nov. 1, 2016 (GB) 1618449.1

(51) Int. Cl. A61F 5/01; A41D 13/00

(52) U.S. Cl. CPC A61F 5/0104; A41D 13/0015; A41D 13/0017; A41D 2400/82; A41D 2300/22; A41D 2500/20; A41D 2400/32; A61F 5/0106

(57) ABSTRACT

A sports garment (1, 6, 10, 60) comprises a body (2, 7) formed of a resilient fabric material and one or more treatment regions (31, 32, 33, 35, 81, 82, 83) including a treatment material having a higher modulus of elasticity than the resilient fabric material, wherein at least a portion of the treatment regions is configured to grip or adhere, in use, at least partially and/or proprioceptively to the body of a wearer.

This invention relates generally to sports garments. More specifically, although not exclusively, this invention relates to sports garments incorporating areas of differing properties to provide support and/or stability to predetermined regions of the body of a wearer of the garment.

It is well known to provide braces, wraps or other support devices to prevent or compensate for injuries in sports activities. Such devices may, for example, protect legs, knees, shoulders, arms, elbows or the neck or torso. Some such devices provide compression to the area by way of tension in a resilient fabric wrapped around the body part and recent efforts have resulted in sports garments, such as leggings and sports tops, incorporating such compression characteristics.

US 2014/0059741 describes a sports garment that imparts a stretching force to particular muscles of the wearer in order to reduce muscle fatigue. This is achieved using bands made of spandex fibre or rubber, which extend along leg muscles and which are mounted on the garment body for imparting a stretching force to the wearer to reduce fatigue.

WO 2016/059579 describes a sports garment for improving comfort and performance in sporting activities. This document is focused on providing areas of compression to certain muscle tissues that present greater oscillations during the practice of sport. The garment includes areas with different levels of resilience which provide different levels of compression to these predetermined areas. The variation in resilience is achieved by depositing silicone on a resilient fabric in the areas where higher compression is desired, thereby reinforcing the resilience in these areas.

The present invention seeks to build on such known sports garment designs and to provide a more effective means of supporting problematic regions of the wearer's body.

Accordingly, a first aspect of the invention provides a sports garment comprising a body formed of a resilient fabric material and one or more treatment regions including a treatment material having a higher modulus of elasticity than the resilient fabric material, wherein at least a portion of the treatment regions is configured to grip or adhere, in use, at least partially and/or proprioceptively to the body of a wearer.

The provision of proprioceptive adhesion or grip enables the garment to be configured to provide an adjunctive therapy, for example similar to the use of kinesiology tape, for improving range of movement, muscular endurance and motor control.

At least a portion of the treatment regions may be configured to grip or adhere to the skin of the wearer, for example to prevent or inhibit movement of the gripped skin as the person moves. Additionally or alternatively, the gripping or adhesion may be configured to protect and/or support a muscle and/or joint of the wearer, e.g. whilst permitting functional muscle movement. The gripping or adhesion may additionally or alternatively be configured to elicit, in use, a sensory response. The gripping or adhesion may additionally or alternatively be configured to move, in use, the fascia away from the muscle and/or reposition superficial tissues (skin and fascia) and/or create space between skin and facsia (e.g. creating one or more areas of low pressure). The gripping or adhesion may additionally or alternatively be configured to reposition joints, e.g. by relieving abnormal muscle tension, and/or to compensate for weak muscles and tendons and/or to improve posture or reduce stress on the spine and/or to reduce strain on supporting muscles and ligaments.

The body may be tubular, e.g. for receiving and/or surrounding or covering the body part of a wearer, such as a leg, arm, neck or torso. The treatment region may circumscribe, in use, the body part of a wearer. The garment may comprise one or more, e.g. at least two, treatment bands, which may have a higher modulus of elasticity than the resilient fabric material. The treatment bands may be formed in or on or secured to the body or resilient fabric material. The treatment bands may extend about the tubular body. The treatment bands may join or merge with, cross or overlap one another, for example to describe the, or one of the treatment regions.

A second aspect of the invention provides a sports garment comprising a tubular body formed of a resilient fabric material and at least two treatment bands having a higher modulus of elasticity than the resilient fabric material and being formed in or on or secured thereto, wherein the treatment bands extend about the tubular body and join, merge, cross or overlap one another to describe a treatment region which circumscribes, in use, the body part of a wearer.

At least one of the treatment regions or bands may be secured to or otherwise formed, deposited or printed on a surface of the body. Preferably, at least one of the treatment regions or bands means is incorporated into or integral with the body, e.g. the resilient fabric material. Each of the treatment bands may have a longitudinal dimension and/or a lateral dimension. At least one or each of the treatment bands may have a higher modulus of elasticity along the longitudinal dimension than along the lateral dimension. Additionally or alternatively, at least one or each of the treatment bands may have a higher modulus of elasticity along the longitudinal dimension than the fabric material, e.g. of the body. At least a portion of at least one of the treatment bands may be configured to adhere, in use, at least partially and/or proprioceptively to the body of a wearer.

The garment, or at least a portion of the treatment regions or at least a portion of at least one of the treatment bands, may include an adhesion, grip or antislip means. The adhesion, grip or antislip means may comprise an adhesive, grip or antislip material. The adhesion, grip or antislip means may have adhesive properties or may exhibit a greater tendency to adhere, e.g. proprioceptively, to the body of a wearer. The adhesion, grip or antislip means may have a higher tack than the resilient fabric material. The one or more treatment regions or bands may comprise an adhesion, grip or antislip material that may provide the adhesion, grip or antislip means. The adhesion, grip or antislip means may be secured to or otherwise formed, deposited or printed on an internal surface of the body or at least one of the treatment regions or bands. Preferably, the adhesion, grip or antislip means is incorporated or woven into or integral with a base fabric or alternatively with the body, e.g. the resilient fabric material, or at least one of the treatment regions or bands.

In embodiments, at least one of the treatment regions or bands, e.g. the adhesion, grip or antislip means, comprises one or more elongate elements, such as one or more filaments, fibres, strands, threads or yarns. The one or more elongate elements may be incorporated, for example stitched, woven, knitted or braided, into a base fabric or into the body, e.g. the resilient fabric material. The one or more elongate elements may be incorporated into the resilient fabric material or into the treatment region(s) or treatment band(s).

The fabric material may comprise an elastane or elastic polyurethane material and a nylon or an aliphatic or semi-aromatic polyamide material. The fabric material may comprise a micro-fibre nylon material, preferably comprising an air textured yarn. The air textured yarn may comprise distorted fibres having increased loft to create air pockets between the fibres. The air pockets may be configured to wick away moisture, for example whilst maintaining warmth. The fabric material may additionally or alternatively include an antimicrobial agent and/or a moisture management finish.

At least one of the treatment regions or bands comprises an elastane backed fabric or base fabric, which may be configured to provide the proprioceptive adhesion. More preferably, at least one of the treatment regions or bands comprises a fabric blend which is different from the fabric material, but which also includes an elastane or elastic polyurethane material and a nylon or an aliphatic or semi-aromatic polyamide material.

At least one of the treatment regions or one or more of the treatment bands may surround and/or extend over an area, joint, muscle or muscle group to be treated. The adhesion, grip or antislip means may extend along and/or across at least one, e.g. each, of the treatment regions or bands. At least two of the treatment bands may converge toward and/or diverge from one another. At least part of at least one of the treatment bands may extend along a helical or spiral path. In embodiments, the garment may comprise a pair of treatment bands that extend about the tubular body along a respective helical or spiral path, for example such that they overlap and/or form a criss-cross pattern. In embodiments, at least two treatment bands extend along opposing helical paths, for example converging toward and diverge from one another along the tubular body.

The garment may comprise a top or shirt covering at least part of the torso of a wearer. The garment may include a treatment region or at least one treatment band extending, in use, over and/or along at least one or each of the rhomboid muscles of a wearer. The garment may include a pair of treatment bands which join, merge, cross or overlap in a region thereof which is over and/or adjacent and/or between, in use, the rhomboid muscles of a wearer. The garment may include a treatment region or at least one treatment band extending, in use, over and/or along and/or beside one or more external abdominal oblique muscles of a wearer. The garment may include a treatment region or at least one treatment band extending, in use, around and/or over the shoulder of a wearer. The garment may include a treatment region or at least one treatment band extending, in use, along and/or over the arm of a wearer. The garment may include a treatment region or at least one treatment band that extending, in use, from a shoulder to a wrist of the wearer.

The garment may include a back portion, e.g. for covering the back of a wearer. The garment may include a front portion, e.g. for covering the front of a wearer. The garment may include one or more arm or sleeve portions, e.g. for covering an arm or a respective arm of a wearer. The garment may include a torso portion, which may include the back portion and/or the front portion and/or arm apertures. The arm or sleeve portions may be joined to and/or may extend from the torso portion or arm apertures thereof.

The garment may include a pair of treatment band portions that join, merge, cross or overlap in a central region of the back portion, for example between the arm or sleeve portions or apertures, e.g. between a lower portion of the join between each arm or sleeve portion and the back portion or between a lower portion of the arm apertures. The garment may include a pair of treatment band portions, each of which may extend from a central portion of a respective side of the front of the garment and extend downwardly toward a central part of the lower edge of the front portion of the garment. The garment may include a treatment band portion that extends along each arm or sleeve portion.

In embodiments, the aforementioned treatment band portions are provided by a single pair of treatment bands. The garment may include a pair of treatment bands each of which extends from a free end of one of the arm or sleeve portions, along the arm or sleeve portion to a front portion of the other (e.g. upper) end of the arm or sleeve portion, e.g. corresponding to a shoulder portion, to the back portion, where the bands join, merge, cross or overlap around a respective outer side of the back portion, to the front portion and down to a central part of the lower edge of the front portion of the garment.

The garment may comprise trousers, pants or bottoms, for example including a pair of leg portions, e.g. for covering the legs of a wearer, which leg portions may be joined together by a pelvic and/or crotch portion, e.g. for covering the pelvis and/or crotch of a wearer. The garment may include a treatment region or at least one treatment band extending, in use, over and/or along and/or around at least part of the iliopsaos and/or pectineus muscles of a wearer. The garment may include a treatment region or at least one treatment band extending, in use, over and/or under and/or around at least part of the gluteus muscles, e.g. the gluteus maximus muscles, of a wearer. The garment may include a treatment region or at least one treatment band extending, in use, over and/or under and/or around at least part of the patella of a wearer. The garment may include a treatment region or at least one treatment band extending, in use, over and/or along and/or around at least part of the calf muscle of a wearer.

The garment may include a pair of band portions on each side of the pelvic portion, which may join, merge, cross and/or overlap, for example in a region thereof which is over and/or along and/or adjacent, in use, the iliopsaos and/or pectineus muscles of a wearer. The garment may include a pair of band portions in each leg portion which join, merge, cross and/or overlap, for example in a region thereof which is under or beneath, e.g. immediately under or beneath, the gluteus maximus muscle of a wearer. The garment may include a pair of band portions in each leg portion which join, merge, cross and/or overlap in a region thereof which is above, e.g. immediately above, the patella of a wearer. The garment may include a pair of band portions in each leg portion which join, merge, cross and/or overlap in a region thereof under or beneath, e.g. immediately under or beneath, the patella of a wearer. The garment may include one or more pairs of band portions in each leg portion which join, merge, cross and/or overlap in a region thereof above, e.g. immediately above, the calf of a wearer.

In embodiments, the aforementioned treatment band portions of the trousers, pants or bottoms are provided by a single pair of treatment bands. In embodiments, the aforementioned treatment band portions of the trousers, pants or bottoms are provided by at least two, e.g. four, pairs of treatment bands. The garment may include first and second pairs of treatment bands, wherein each band of each pair extends from a respective one of the front and back of the pelvic portion, to join, merge, cross or overlap at one side of the front of the pelvic portion, around to the rear side of one of the leg portions to join, merge, cross or overlap in an upper part of the leg portion, around to the front side of the leg portion to join, merge, cross or overlap in a central part of the leg portion, around the leg portion to join, merge, cross or overlap a second time at the front side of the leg portion immediately below or beneath the central part of the leg portion and around to the rear side of the leg portion where the bands join, merge, cross or overlap and extend downwardly to a lower, free edge of the leg portion.

Preferably the garment may feel and/or appear from the exterior surface identical or similar to a regular equivalent garment. In an embodiment, the or each treatment region is integral with the body, e.g. the or each treatment region is weaved or knitted with or into or is stitched to the body. The innermost surface of the treatment region having grip or antislip means or function, whereas the exterior surface does not. Where the grip or antislip means or function is provided by an elastomer weave, for example an elastane weave, the proportion of the treatment region comprising elastomer (elastane) may be higher at the innermost surface of the garment than at the outer surface. The elastomer (elastane) may be provided at or towards the innermost surface. For example, the or each treatment region may comprise a fabric which is relatively elastomer-rich (elastane-rich) on an inner surface and relatively elastomer-poor (elastane-poor) at an outer surface. The or each treatment region may comprise a first innermost zone which is relatively elastomer-rich (elastane-rich) and a second, relatively outer region which is relatively elastomer-poor (elastane-poor).

For the avoidance of doubt, any of the features described herein apply equally to any aspect of the invention. Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible. For the avoidance of doubt, the terms “may”, “and/or”, “e.g.”, “for example” and any similar term as used herein should be interpreted as non-limiting such that any feature so-described need not be present. Indeed, any combination of optional features is expressly envisaged without departing from the scope of the invention, whether or not these are expressly claimed. The applicant reserves the right to change any originally filed claim or file any new claim accordingly, including the right to amend any originally filed claim to depend from and/or incorporate any feature of any other claim although not originally claimed in that manner.

Embodiments of the invention will now be described by way of example only with reference to the accompanying drawings in which:

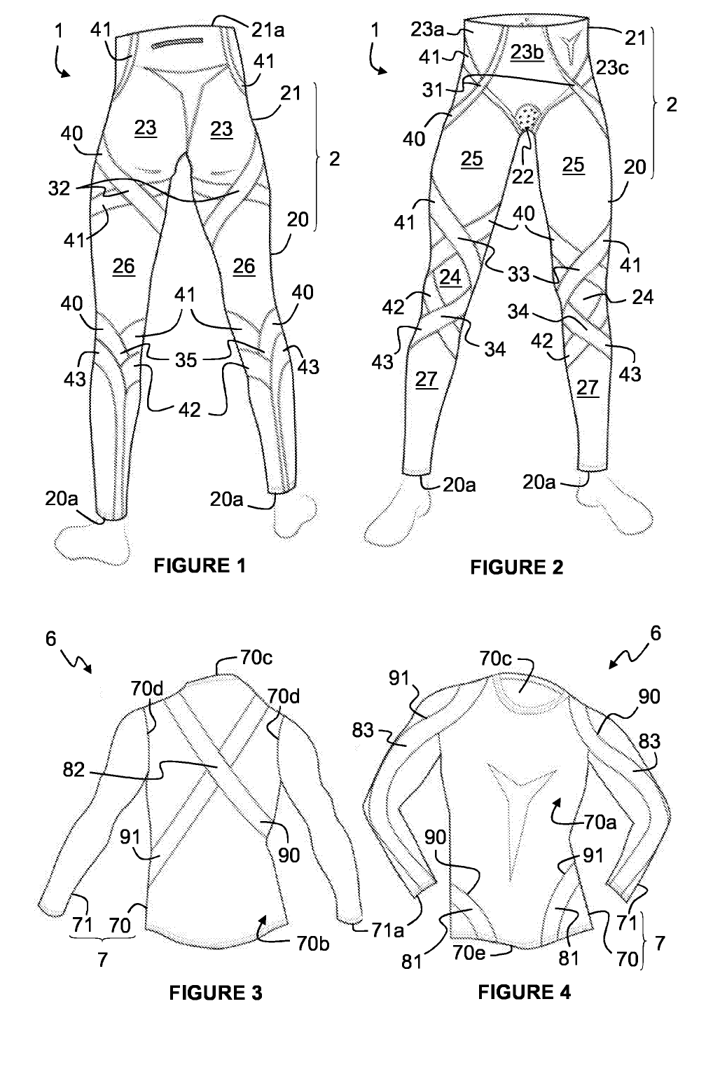

FIG. 1 is a rear view of a trouser-type sport garment according to an embodiment of the invention adapted for men;

FIG. 2 is a front view of the trouser-type sport garment of FIG. 1;

FIG. 3 is a rear view of a long-sleeved shirt-type sport garment according to an embodiment of the invention adapted for men;

FIG. 4 is a front view of the long-sleeved shirt-type sport garment of FIG. 3;

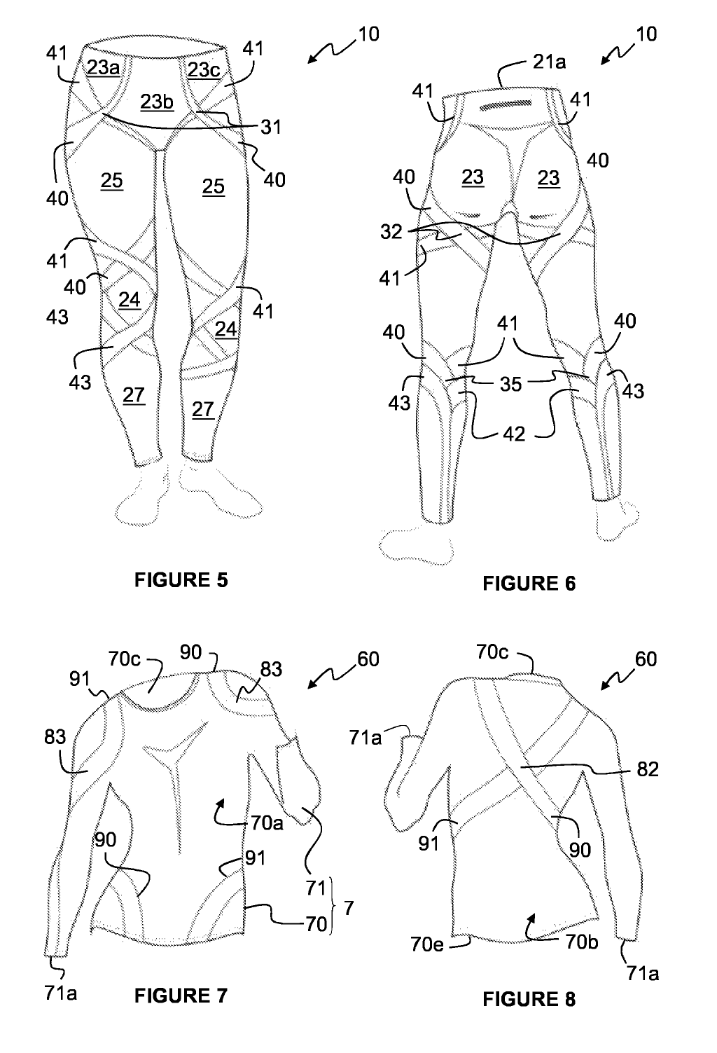

FIG. 5 is a front view of a trouser-type sport garment according to an embodiment of the invention adapted for women;

FIG. 6 is a rear view of the trouser-type sport garment of FIG. 5;

FIG. 7 is a front view of a long-sleeved shirt-type sport garment according to an embodiment of the invention adapted for women; and

FIG. 8 is a rear view of the long-sleeved shirt-type sport garment of FIG. 7.

Referring now to FIGS. 1 and 2, there is shown a trouser-type sport garment 1 according to an embodiment of the invention, which is specifically adapted for men. The garment includes a body 2 formed of resilient fabric material and a plurality of treatment regions 31, 32, 33, 34, 35 described by treatment bands 40, 41, 42, 43 formed of a treatment material having a higher modulus of elasticity than the resilient fabric material of the body 2.

The body 2 includes a pair of tubular leg portions 20 for covering the legs of a wearer (not shown) and a tubular pelvic portion 21 for covering the pelvis and crotch of a wearer (not shown). The leg portions 20 are open at their lower ends 20a and are joined at their upper ends by the pelvic portion 21 in the normal way. The pelvic portion 21 is open at its upper end 21a and includes a central, lower crotch region 22.

Each leg portion 20 includes a first pair of treatment bands 40, 41 which extend from the pelvic portion 21 to the lower end 20a along a spiral or helical path in opposite directions such that they join one another in various areas. A first of the treatment bands 40 extends from the front side of the top end 21a of the pelvic portion 21 above the leg portion 20, while a second of the treatment bands 41 extends from the back side of the top end 21a above the leg portion 20. The second treatment band 41 wraps around the pelvic portion 21 to the front side 21a and immediately below the crotch region 22. The first treatment band 40 extends along an opposing helical path to that which the first treatment band 40 extends, such that it crosses over, or joins, the second treatment band 41 in a treatment region 31 designed to cover to the iliopsaos and/or pectineus muscles of a wearer (not shown).

The treatment bands 40, 41 then extend over the back side of the leg portion 20, with the first treatment band 40 crossing the second treatment band 41 again at the back side 20d immediately under a region 23 of the body 2 designed to cover the gluteus maximus muscle of a wearer (not shown). The treatment bands 40, 41 then wrap back around to the front side of the leg portion 20, where the second treatment band 41 crosses the first treatment band 40 immediately above a region 24 designed to cover the patella of a wearer (not shown). Finally, the treatment bands 40, 41 wrap around the leg portion 20 to the back side thereof, where the first treatment band 40 crosses the second treatment band 41 above a region designed to cover the calf muscle of a wearer (not shown).

In this embodiment, each leg portion 20 also includes a second pair of treatment bands 42, 43. Each of which extends along an opposing helical path from beneath a respective one of the first and second treatment bands 40, 41, under the joined portion immediately above the patella region 24 at each lateral side of the leg portion 20. A third treatment band 42 extends under a fourth treatment band 43 immediately beneath the patella region 24 and wraps around the leg portion 20 to the back side thereof, where the fourth treatment band 43 joins the first, second and third treatment bands 40, 41, 42 and extends downwardly along the region designed to cover the calf muscle of a wearer (not shown) to the lower end 20a of the leg portion 20.

Thus, the treatment bands 40, 41, 42, 43 describe various treatment regions including and extending from a iliopsaos or pectineus treatment region 31, a gluteus treatment region 32, upper and lower patella treatment regions 33, 34 and a calf treatment region 35. In addition, the treatment bands 40, 41, 42, 43 also delineate fabric segments of the body 2, including the gluteus region 23, which also covers a portion of the lower back of a wearer (not shown), three frontal pelvic regions 23a, 23b, 23c, thigh regions 25, hamstring regions 26, the aforementioned patella regions 24, and shin regions 27.

In this embodiment, both the body 2 and treatment bands 40, 41, 42, 43 include a fabric material which includes blend of elastane and nylon. The fabric material of the body is in the form of an air textured yarn with distorted fibres having increased loft to create air pockets between the fibres. The fibres are distorted by blasting them intermittently with jets of air to provide yarns with more loft. The air pockets are configured to wick away moisture whilst maintaining warmth. The fabric material may also include an antimicrobial agent and/or a moisture management finish, but the aforementioned wicking effect mitigates microbial growth.

The fabric of the treatment bands 40, 41, 42, 43 is an elastane backed fabric configured to provide the proprioceptive adhesion and exhibiting, together with the base fabric, a higher modulus of elasticity than the fabric material of the body 2. In this embodiment, different fabrics are also used for different regions of the body 2. More specifically, the gluteus region 23 and the outer pelvic regions 23a, 23c are formed of a fabric that provides core back support, while the thigh, hamstring and shin regions 25, 26, 27 are formed of a fabric selected for optimal cooling and the crotch and patella regions 22, 24 are formed of a warming and venting fabric material. Other materials with different properties may also be beneficial, as the skilled person would appreciate.

In use, the helical, crossing treatment bands 40, 41, 42, 43 adhere, or at least provide proprioceptive adhesion, to the body of a wearer (not shown) and their higher modulus of elasticity provides support to the hip, gluteus muscles and knees of the wearer (not shown) when engaged in a sporting activity. This arrangement functions in a similar manner to kinesiology tape, which is an established rehabilitation technique used to facilitate the natural healing process, whilst providing support and stability to joints and muscles in the desired regions or quadrants.

Studies have shown that kinesiology tape provides an effective adjunctive therapy for improving range of movement, muscular endurance and motor control. Recent research has also shown improvements in joint position sense, symptoms of patellofemoral syndrome, pain and daily limitations. The present invention can be configured to provide treatment bands 40, 41, 42, 43 which mimic strapping techniques used by physiotherapists and athletic trainers.

Turning now to FIGS. 3 and 4, there is shown a long-sleeved shirt-type sport garment 6 according to an embodiment of the invention, which is also specifically adapted for men. The garment includes a body 7 formed of resilient fabric material and a plurality of treatment regions 81, 82, 83 described by treatment bands 90, 91 formed of a treatment material having a higher modulus of elasticity than the resilient fabric material of the body 7. The treatment bands 90, 91 are formed of the same materials as the treatment bands 40, 41, 42, 43 described above in relation to the trouser-type sport garment 1. The fabric material of the body 7 is selected for optimal cooling in this embodiment, although it is envisaged that certain areas may include different materials as with the trouser-type sport garment 1 described above.

The body 7 includes a torso portion 70 and a pair of arm or sleeve portions 71. The torso portion 70 includes a front 70a, a back 70b, a neck hole 70c, arm holes 70d and an open lower end 70e. The arm portions 71 are open at their lower ends 71a with their upper ends joined to the arm holes 70d of the torso portion 70 in the normal way.

In this embodiment, the garment 6 includes two treatment bands 90, 91, each of which extends along a spiral or helical path from the open lower end 70e up the torso portion 70 and down a respective one of the arm portions 71 to the lower end 71a thereof. A first treatment band 90 starts from the open lower end 70e at the front side 70a of the torso portion 70 to the wearer's right of centre, wraps around the torso portion 70 to the back side 70b and over the wearer's left shoulder and down the left arm portion 71 to the lower end 71a thereof. A second treatment band 91 starts from the open lower end 70e at the front side 70a of the torso portion 70 to the wearer's left of centre, wraps around the torso portion 70 to the back side 70b and over the wearer's right shoulder and down the right arm portion 71 to the lower end 71a thereof.

The initial part of each treatment band 90, 91 runs along a region designed to curve around the wearer's abdominal oblique muscles, thereby providing an abdominal oblique treatment region 81. The portion of each treatment band 90, 91 running across the back 70b of the torso portion 70 is designed to cover the wearer's rhomboid muscles and the first treatment band 90 crosses the second treatment band 91 between the wearer's shoulders, thereby providing a rhomboid treatment region 82. The portion of each treatment band 90, 91 that runs over the shoulder and down the arm portion 71 grip and support the shoulder, elbow and wrist of the wearer, thereby providing shoulder and arm treatment regions 83.

As described above with respect to the trouser-type sport garment 1, the treatment bands 90, 91 adhere proprioceptively to the wearer's body and support the abdominal oblique muscles, the rhomboid muscles, shoulders, elbows and wrists of the wearer during a sporting activity. This arrangement is intended to function in a similar manner to the use of kinesiology tape as a rehabilitation technique for facilitate the natural healing process, whilst providing support and stability to joints and muscles in the desired areas.

FIGS. 5 and 6 illustrate a trouser-type sport garment 10, which is a variation of the trouser-type sport garment 1 of FIGS. 1 and 2 designed specifically for women, wherein similar reference numbers depict similar features that will not be described further herein. The garment 10 according to this embodiment differs from the garment 1 of FIGS. 1 and 2 only in the positioning and width of the treatment bands 40, 41, 42, 43.

Similarly, FIGS. 7 and 8 illustrate a long-sleeved shirt-type sport garment 60, which is a variation of the long-sleeved shirt-type sport garment 6 of FIGS. 3 and 4 designed specifically for women, wherein similar reference numbers depict similar features that will not be described further herein. The garment 60 according to this embodiment differs from the garment 6 of FIGS. 2 and 3 only in the positioning and width of the treatment bands 90, 91.

The present invention may be applied to other types of garment or to only parts of the garments 1, 10, 6, 60 illustrated and described herein. In addition, it will be appreciated by those skilled in the art that several variations to the aforementioned embodiments are envisaged without departing from the scope of the invention. For example, more or less treatment bands 40, 41, 42, 43, 90, 91 may be used to provide similar configurations to those described and shown in the drawings.

Similarly, the treatment bands 40, 41, 42, 43, 90, 91 may be configured differently, for example covering different regions and/or crossing in a different manner and/or in different areas. Advantageously, the treatment bands 40, 41, 42, 43, 90, 91 may be configured to mimic various other kinesiology taping techniques.

In addition, the materials and weaving techniques described above may vary. The proprioceptive adhesion may be provided by some other means, for example a material (e.g. silicone or other material) may be deposited on an inner surface of the garment, for example at or along the treatment bands 40, 41, 42, 43, 90, 91 or treatment regions 31, 32, 33, 34, 35, 81, 82, 83.

It will also be appreciated by those skilled in the art that any number of combinations of the aforementioned features and/or those shown in the appended drawings provide clear advantages over the prior art and are therefore within the scope of the invention described herein.

1-15. (canceled)

16. A sports garment comprising a body formed of a resilient fabric material and one or more treatment regions including a treatment material having a higher modulus of elasticity than the resilient fabric material, wherein at least a portion of the treatment regions is configured to grip or adhere, in use, at least partially and/or proprioceptively to the body of a wearer.

17. The sports garment according to claim 16, wherein at least a portion of the treatment regions comprises grip means woven into a base fabric.

18. The sports garment according to claim 17, wherein the grip means comprises a higher tack than each of the base fabric and the resilient fabric material.

19. The sports garment according to claim 17, wherein the grip means comprises an elastane material.

20. The sports garment according to claim 17, wherein the body is tubular and the one or more treatment regions comprise at least two treatment bands formed in or on or secured to the body, the treatment bands comprising the grip means, extending about the tubular body and joining, crossing or overlapping one another to describe a treatment region which circumscribes, in use, the body part of a wearer.

21. The sports garment according to claim 20, wherein the at least two treatment bands extend along opposing helical paths converging toward and diverge from one another along the tubular body.

22. The sports garment according to claim 21, wherein the treatment bands join or cross in a region located, in use, between the rhomboid muscles of a wearer.

23. The sports garment according to claim 20, wherein at least one of the treatment bands extends, in use, over and/or along and/or beside one or more external abdominal oblique muscles of a wearer.

24. The sports garment according to claim 21, wherein at least one of the treatment bands extends, in use, over and/or along and/or beside one or more external abdominal oblique muscles of a wearer.

25. The sports garment according to claim 20, wherein at least one treatment bands extends, in use, around and/or over the shoulder and/or along and/or over the arm of a wearer.

26. The sports garment according to claim 21, wherein at least one treatment bands extends, in use, around and/or over the shoulder and/or along and/or over the arm of a wearer.

27. The sports garment according to claim 20 comprising trousers with a pair of leg portions for covering the legs of a wearer joined together by a pelvic portion for covering the pelvis and crotch of a wearer.

28. The sports garment according to claim 27, wherein at least one of the treatment bands extends, in use, over and/or along and/or around at least part of the iliopsaos and/or pectineus muscles of a wearer.

29. The sports garment according to claim 27, wherein at least one of the treatment bands extends, in use, over and/or under and/or around at least part of the gluteus muscles of a wearer.

30. The sports garment according to claim 28, wherein at least one of the treatment bands extends, in use, over and/or under and/or around at least part of the gluteus muscles of a wearer.

31. The sports garment according to claim 27, wherein at least one of the treatment bands extends, in use, over and/or under and/or around at least part of the patella of a wearer.

32. The sports garment according to claim 27, wherein at least one of the treatment bands extends, in use, over and/or along and/or around at least part of the calf muscle of a wearer.

33. The sports garment according to claim 27, wherein the at least two treatment bands comprise a pair of band portions on each side of the pelvic portion that join or cross in a region thereof which is over or adjacent the iliopsaos and/or pectineus muscles of a wearer, a pair of band portions in each leg portion which join or cross in a region thereof which is under or beneath the gluteus maximus muscle of a wearer, a pair of band portions in each leg portion which join or cross in a region thereof which is above the patella of a wearer, a pair of band portions in each leg portion which join or cross in a region thereof under the patella of a wearer and one or more pairs of band portions in each leg portion which join or cross in a region thereof that is above the calf of a wearer.

34. A sports garment comprising a tubular body formed of a resilient fabric material and at least two treatment bands having a higher modulus of elasticity than the resilient fabric material and being formed in or on or secured thereto, wherein the treatment bands extend about the tubular body and join, merge, cross or overlap one another to describe a treatment region which circumscribes, in use, the body part of a wearer.

35. The sports garment according to claim 34, wherein at least one of the at least two treatment bands is incorporated into or integral with the body.
